(19) United States
(12) Patent Application Publication
(10) Pub. No.: US 2005/0244374 A1
(43) Pub. Date: Nov. 3, 2005

(54) Enveloped miroorganism

(76) Inventors: Werner Goebel, Gerbrunn (DE); Ulf R. Rapp, Wurzburg (DE); Hans-Harald Sedlacek, Marburg (DE); Joachim Fensterle, Hochberg (DE); Ivaylo Gentschev, Kist (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/504,944

(22) PCT Filed: Feb. 13, 2003
(86) PCT No.: PCT/DE03/00470

§ 371 Date: May 23, 2005

(30) Foreign Application Priority Data
Feb. 14, 2002 (DE) 10206325.7

(51) Int. Cl. A61K 48/00; C12N 1/21; C12N 15/74

(52) U.S. Cl. 424/93.2; 435/252.3; 435/471; 424/93.4

(57) ABSTRACT

The invention relates to an enveloped microorganism in whose genome the following components are inserted and can be expressed: I) a nucleotide sequence that encodes a directly or indirectly, antiproliferatively or cytotoxically active expression product or a plurality of said expression products, II) a nucleotide sequence that encodes or is constitutively active for a blood plasma protein under the control of a activation sequence that can be activated in the microorganism, III) optionally, a nucleotide sequence that encodes or is constitutively active for a cell-specific ligand under the control of an activation sequence that can be activated in the microorganism, IV) a nucleotide sequence for a transport system that induces expression of the expression products of components I) and II) and optionally III) on the outer surface of the microorganism or that induces secretion of the expression products of component I) and expression of component II) and optionally component III) and that is preferably constitutively active, V) optionally a nucleotide sequence for a protein used for lysis of the microorganism in the cytosol of mammalian cells and for the intracellular release of plasmids with at least one or more components I) and VI) contained in the lysed microorganism, and VI) an activation sequence that can be activated in the microorganism, and/or that is tissue-specific, tumor cell-specific, function-specific or not cell-specific, for expressing component I). The inventive microorganism is further characterized in that any of components I) to VI) can be present either single or several times, and can be either identical or different.

### FIELD OF THE INVENTION

The invention relates to a microorganism with foreign nucleotide sequences, by means of which antiproliferatively or cytotoxically acting expression products can be expressed, and to the use of such microorganisms for the production of pharmaceutical compositions, to a plasmid and a method for the production of such a microorganism, and to the uses of such microorganisms.

### BACKGROUND OF THE INVENTION AND PRIOR ART

Virulence-reduced microorganisms, such as genetically modified viruses, or virulence-attenuated bacteria gain increasing importance as carriers of foreign nucleic acid sequences to be used in the gene therapy.

For the gene therapy, the foreign nucleic acids are either inserted in vitro into tissue cells, and these cells are administered to the patient, or the microorganisms are injected to the patient, expecting that the microorganisms transfer as gene ferries the foreign nucleic acid into the desired tissue cell.

Microorganisms are particles. After injection into an organism, these particles are mainly received by the so-called reticuloendothelial system. In order to achieve against this elimination mechanism nevertheless an enrichment of the microorganisms used as gene ferries in a target tissue, the microorganisms are provided with cell-specific ligands. Up to now, in spite of this provision, the elimination of the microorganisms by the reticuloendothelial system could only slightly be reduced.

An essential research aim of the gene therapy is the therapy of proliferative diseases—such as tumors, leukemias, chronic inflammations, autoimmune diseases and rejections of transplanted organs, the treatment of which is still insufficient, in spite of all successes of the medicament therapy. For instance, in spite of all successes of surgery, radiotherapy, chemotherapy and also immune therapy for the treatment of tumors, there could not be achieved up to now a healing of advanced tumors of the head and the neck, the central nervous system, the mammary gland, the lung, the gastrointestinal tract, the liver, the pancreas, the kidney, the skin, the ovaries and the prostate.

The reasons for this poor success of the tumor therapy are manifold and not yet comprehensively known. To the main reasons, however, belong, i) before (primarily) existing resistances of the tumor cells against the in vivo achievable concentrations of chemotherapeutic agents, of irradiations or against immunotherapeutic agents; ii) resistances against the respective therapeutic agent generated in response to the therapy. These induced so-called secondary resistances are caused by the genetic variability of the tumor cells permitting them to avoid the effects of the tumor therapeutic agents by the development of resistance mechanisms; iii) pharmacokinetic and/or pharmacodynamic insufficiencies of the up to now available tumor therapeutic agents, due to which the concentration of the respective tumor therapeutic agent, irrespective of whether there are primary tumors, recidivations or metastases, is absolutely too small to eliminate the tumor. To these insufficiencies of the tumor therapeutic agents belong, iv) a too high distribution volume; v) the insufficient enrichment at the tumor or at the tumor cells; vi) the insufficient penetration capability in the tumor; and/or vii) the toxic effect on the total organism, which limits an increase of the dose for an increased enrichment at the tumor.

In the past, different methods were used for trying to enrich tumor therapeutic agents at the tumor.

Tumor cell-specific ligands, for instance antibodies or the fission products thereof, coupled to cytostatics, to antitumorally acting cytokines, to cytotoxic proteins, or to isotopes, did lead to an enrichment of the cytotoxic active substances at the tumor, compared to the normal tissue, however this enrichment was in the by far most cases not sufficient for a therapy of the tumor (survey: Sedlacek et al., Contributions to Oncology 43:1-145, 1992; Carter, Nature Reviews Cancer 1:118-129, 2001).

As a consequence, amplification systems have been designed, by means of which the concentration of the respective active substance at the tumor could be increased.

An amplification sequence had the aim to introduce such enzymes in the tumor, which were not generally accessible or foreign in the remaining body, and which in turn could convert or split in the tumor a non-toxic pre-stage of a cytostatic into the cytotoxically active cytostatic. The introduction of the enzymes into the tumor was performed either by administration of tumor cell-specific ligands, coupled to these enzymes (for instance in the form of the antibody derived enzyme-mediated prodrug therapy; ADEPT), or by the administration of genes for these enzymes by means of tumor cell-specific or not specific vectors (gene derived enzyme-mediated prodrug therapy; GDEPT) (Sedlacek et al., Contributions to Oncology 43:1-145, 1992; Sedlacek, Critical Reviews in Oncology/Hematology 37:169-215, 2001; McCormick, Nature Reviews Cancer 1:130-141, 2001; Carter, Nature Reviews Cancer 1:18-129, 2001).

The prior clinical investigations with ADEPT or GDEPT have furnished insufficient therapeutic results, however. As essential problems could be identified, i) the immunogenicity of a foreign enzyme; ii) the relatively small tumor localization rate of an antibody-enzyme conjugate (ADEPT); iii) the technical difficulties to produce fusion proteins from a humanized antibody with a human enzyme in a sufficiently large amount at acceptable costs; iv) the lacking tumor penetration of the antibody-enzyme conjugates or the gene vectors; and v) the too small transduction rate in vivo, i.e. the number of tumor cells of a tumor node, into which the genes for the enzyme could be expressed, was too small for a tumor-therapeutic effectivity.

Another amplification system is based on the induction of an immune reaction against tumor cells, in the course of which specific antibody-forming cells and cytotoxic cells proliferate. For the induction of an immune reaction, tumor antigens are administered in a suitable preparation. It is the aim to break the immune tolerance against the tumor, this immune tolerance obviously existing in tumor patients, and/or the resistance of the tumor against the own immune reaction.

Within the last decades, numerous technical variations of the tumor vaccination were clinically investigated by combination of different tumor antigens with adjuvants, however without achieving the desired break-through in the tumor therapy. New approaches, such as the administration of combinations of immunogenic tumor-specific antigens with new adjuvants, or of dendritic cells, loaded with tumor-specific antigens, or of nucleotide sequences that encode tumor-specific antigens, have resulted in first promising clinical results, however up to now there cannot be seen a break-through in the tumor therapy here, too.

A technique has been developed to express expression products of nucleic acid sequences introduced into bacteria on the cell membrane of these bacteria or to have them secreted by these bacteria. The basis of this technique is the Escherichia coli hemolysin system hlyAs, which represents the prototype of a type I secretion system of gram-negative bacteria. By means of the hlyAs, secretion vectors were developed, which permit an efficient discharge of protein antigens in Salmonella enterica, Yersinia enterocolitica and Vibrio cholerae. Such secretion vectors contain the cDNA of an arbitrary protein antigen coupled to the nucleotide sequence for the hlyA signal peptide, for the hemolysin secretion apparatus, hlyB and hlyD and the hly-specific promoter. By means of this secretion vector, a protein can be expressed on the surface of this bacterium. Thus genetically modified bacteria induce as vaccines a considerably higher immune protection than bacteria, in which the protein expressed by the introduced nucleic acid remains intracellularly (Donner et al., EP 1015023 A, Gentschev et al., Gene 179:133-140, 1996; Vaccine 19:2621-2618, 2001; Hess et al., PNAS 93:1458-1463, 1996). The drawback of this method is however that by using the hly-specific promoter the amount of the protein expressed on the outer surface of the bacterium is extremely small.

A technique for the introduction of plasmid DNA into mammalian cells by carrier bacteria such as Salmonella and Listeria monocytogenes has been developed. Genes contained in these plasmids could be expressed in the mammalian cells, even when they were under the control of a eukaryontic promoter. Plasmids were introduced into Listeria monocytogenes germs, said plasmids containing a nucleotide sequence for an arbitrary antigen under the control of an arbitrary eukaryontic promoter. By introduction of the nucleotide sequences for a specific lysis gene, it was achieved that the Listeria monocytogenes germs dissolve in the cytosol of the antigen-presenting cell and release their plasmids, which then leads to expression, processing and presentation of the plasmid-coded proteins and clearly increases the immunogenicity of these proteins (Dietrich et al., Nat. Biotechnol. 16:181-185, 1998; Vaccine 19:2506-2512, 2001).

Virulence-attenuated variants of bacteria settling intracellularly have been developed. For instance such variants of Listeria monocytogenes, Salmonella enterica sv. typhimurium and typhi and BCG were already used as well tolerated live vaccines against typhus and tuberculosis. These bacteria including their attenuated mutants are generally immune stimulating and can trigger a fair cellular immune response. For instance L. monocytogenes stimulates to a special degree via the activation of TH1 cells the proliferation of cytotoxic lymphocytes. These bacteria supply secerned antigens directly into the cytosol of antigen-presenting cells (APC; macrophages and dendritic cells), which in turn express the costimulating molecules and trigger an efficient stimulation of T cells. The listeriae were in part degraded in phagosomal compartments, and the antigens produced by these carrier bacteria can therefore on the one hand be presented via MHC class II molecules and thus lead to the induction of T helper cells. On the other hand, the listeriae replicate after release from the phagosome in the cytosol of APCs; antigens produced and secerned by these bacteria are therefore preferably presented via the MHC class I pathway, thereby CTL responses against these antigens being induced. Furthermore, it could be shown that by the interaction of the listeriae with macrophages, natural killer cells (NK) and neutrophilic granulocytes, the expression of such cytokines (TNF-alpha, IFN-gamma, Il-2, IL-12; Unanue, Curr. Opin. Immunol. 9:35-43, 1997; Mata and Paterson, J. Immunol. 163:1449-14456, 1999) is induced, for which an antitumoral effectivity was detected. For instance, by the administration of L. monocytogenes, which were transduced for the expression of tumor antigens, the growth of experimental tumors could antigen-specifically be inhibited (Pan et al., Nat. Med. 1:471-477, 1995; Cancer Res. 59:5264-5269, 1999; Voest et al., Natl. Cancer Inst. 87:581-586, 1995; Beatty and Paterson, J. Immunol. 165:5502-5508, 2000). Virulence-attenuated Salmonella enterica strains, into which nucleotide sequences that encode tumor antigens have been introduced, could cause as tumor antigen-expressing bacterial carriers after oral administration a specific protection against different experimental tumors (Medina et al., Eur. J. Immunol. 30:768-777, 2000; Zoller and Christ, J. Immunol. 166:3440-3450, 2001; Xiang et al., PNAS 97:5492-5497, 2000). Recombinant Salmonella strains were also effective as prophylactic vaccines against virus infections (HPV) (Benyacoub et al., Infect. Immun. 67:3674-3679, 1999) and for the therapeutic treatment of a mouse tumor immortalized by a tumor virus (HPV) (Revaz et al., Virology 279:354-360, 2001). For the systemic tumor therapy, Salmonella strains were selected, which settle on specifically selected tumor tissues (Murray et al., J. Bacteriol. 183:5554-5564, 2001). Into these Salmonella strains as well as into Escherichia coli strains, nucleotide sequences that encode selected enzymes were introduced, and these bacterial carriers were successfully used for GEDPT in vitro as well as in vivo in experimental tumor systems (Pawelek et al., Cancer Res. 57:4537-4544, 1997).

Inflammation tissues and in particular tumor tissues are characterized by an increased angiogenesis in most cases chaotically proceeding in the tumor. In these newly formed vessels, soluble as well as particulate substances can be enriched, provided they have a low distribution volume and thus a relatively long blood half-life. This enrichment (also designated passive targeting) can be used for therapeutic methods (Sedlacek, Critical Reviews in Oncology/Hematology 37:169-215, 2001).

### TECHNICAL OBJECT OF THE INVENTION

It is the object of the present invention to provide a pharmaceutical composition, which has an increased effectiveness in the treatment of proliferative diseases, in particular in the tumor therapy.

Basic Concept of the Invention and Findings the Invention is Based on.

For achieving the above technical object, the invention teaches an enveloped microorganism, in whose genome the following components are inserted and can be expressed: I) a nucleotide sequence that encodes a directly or indirectly, antiproliferatively or cytotoxically active expression product or a plurality of said expression products; II) a nucleotide sequence that encodes or is constitutively active for a blood plasma protein under the control of an activation sequence that can be activated in the microorganism; III) optionally, a nucleotide sequence that encodes or is constitutively active for a cell-specific ligand under the control of an activation sequence that can be activated in the microorganism; IV) a nucleotide sequence for a transport system that induces expression of the expression products of components I) and II) and optionally III) on the outer surface of the microorganism or that induces secretion of the expression products of component I) and expression of component II) and optionally component III) and that is preferably constitutively active; V) optionally a nucleotide sequence for a protein used for lysis of the microorganism in the cytosol of mammalian cells and for the intracellular release of plasmids with at least one or more components I) and VI) contained in the lysed microorganism; and VI) an activation sequence that can be activated in the microorganism, and/or that is tissue-specific, tumor cell-specific, function-specific or not cell-specific, for expressing component I), any of components I) to VI) being able to be present either single or several times, and either identical or different.

For the purpose of the invention, preferably enveloped microorganisms as carriers for gene information and the use of said enveloped microorganisms for the prophylaxis and therapy of a proliferative disease are described. The invention is based on the following experiences and technical developments.

Subject matter of the invention are therefore preferably enveloped microorganisms as carriers for nucleotide sequences for the treatment of proliferative diseases, the following components having been inserted into the microorganisms: I) at least one nucleotide sequence that encodes at least one directly or indirectly, antiproliferatively or cytotoxically active expression product; II) at least one nucleotide sequence that encodes at least one blood plasma protein under the control of at least one activation sequence that can be activated in the microorganism; III) optionally, at least one nucleotide sequence that encodes at least one cell-specific ligand under the control of at least one activation sequence that can be activated in the microorganism; IV) at least one nucleotide sequence for at least one transport system that makes possible the expression of the expression products of components I) and II) and III) on the outer surface of the microorganism or the secretion of component I), II) and III); V) optionally at least one nucleotide sequence for at least one protein used for lysis of the microorganism in the cytosol of mammalian cells and for the intracellular release of plasmids contained in the lysed microorganism; and VI) at least one activation sequence what can be activated in the microorganism or at least one tissue-specific, tumor cell-specific or not cell-specific activation sequence, for expressing component I).

### PREFERRED EMBODIMENTS OF THE INVENTION

Component I).

Component I) is at least one nucleotide sequence that encodes at least one directly or indirectly, antiproliferatively or cytotoxically active expression product. Directly, antiproliferatively active expression products in the meaning of the invention are for instance interferons, such as IFN-alpha, IFN-gamma, IFN-beta, interleukins, which inhibit immune cells or tumor cells, such as IL-10, IL-12, proapoptotic peptides or proteins, such as TNF-alpha, fas ligand, TNF-related apoptosis inducing ligand (TRAIL), antibodies or fragments of antibodies, which act inhibitingly on or cytotoxically for an immune cell, a tumor cell or a cell of the tissue, from which the tumor originates, such as antibodies directed against i) a tumor-associated or tumor-specific antigen, ii) an antigen against lymphocytes, such as against the T cell receptor, the B cell receptor, the receptor for the C40 ligand, the B7.1 or B7.2, the receptor for an interleukin, such as IL-1, -2, -3, -4, -5, -6, -7, -8, -9, -10, -11, -12, -13, -14, -15 or -16, the receptor for an interferon or the receptor for a chemokine, for instance for RANTES, MCAF, MIP-alpha, MIP-beta, IL-8, MGSA/Gro, NP-A-2 or IP-10, iii) a tissue-specific antigen, such as against a tissue-specific antigen of the cells of mammary glands, kidneys, nevi, prostate, thyroid glands, tunica mucosa gastris, ovaries, cervix, vesica urinaria, an antiproliferatively active protein, such as the retinoblastoma protein (pRb=p110), or the related p107 and p130 proteins, or antiproliferatively active mutants of these proteins, the p53 protein and analogous proteins or antiproliferatively active mutants of these proteins, the p21 (WAF-1) protein, the p27 protein, the p16 protein, the GAAD45 protein, antiproliferatively active proteins of the Bcl2 family, such as bad or bak, cytotoxic proteins, such as perforin, granzyme, oncostatin, an antisense RNA or a ribozyme, specific for an mRNA, which participates in the growth or the proliferation of a cell, for instance specific for the mRNA that encodes a receptor, for a signal-transmitting enzyme, for a protein, which participates in the cell cycle, for a transcription factor or for a transport protein. Indirectly, proliferatively active proteins are for instance inductors of acute inflammations and immune reactions, such as chemokines like RANTES (MCP-2), monocyte chemotactic and activating factor (MCAF), IL-8, macrophage inflammatory protein-1 (MIP-1-alpha, -beta), neutrophil activating protein-2 (NAP-2), interleukins, such as IL-1, IL-2, IL-3, IL-4, IL-5, human leukemia inhibiting factor (LIF), IL-6, IL-7, IL-9, IL-11, IL-13, IL-14, IL-15, IL-16, cytokines, such as GM-CSF, G-CSF, M-CSF, enzymes for the activation or fission of the inactive pre-stage of a cytotoxic substance into a cytotoxic substance, said enzymes being an oxidoreductase, a transferase, a hydrolase or a lyase. Examples for such enzymes are β-glucuronidase, β-galactosidase, glucose oxidase, alcohol dehydrogenase, lactoperoxidase, urokinase, tissue plasminogen-activator carboxy peptidase, cytosine deaminase, deoxycytidine kinase, thymidine kinase, lipase, acidic phosphatase, alkaline phosphatase, kinase, purine nucleoside phosphorylase, glucose oxidase, lactoperoxidase, lactate oxidase, penicillin V amidase, penicillin G amidase, lisozyme, β-lactamase, aminopeptidase, carboxypeptidase A, B or G2, nitroreductase, cytochrome P450 oxidase. According to the invention the enzyme can originate from a virus, a bacterium, a yeast, a mollusk, an insect or a mammal. Preferably such enzymes are used, which originate from man. Furthermore, such nucleic acid constructs are preferred in the meaning of the invention, which encode a fusion product of a cell-specific ligand with an enzyme, and/or proteins, which inhibit angiogenesis, for instance plasminogen activator inhibitor-1 (PAI-1), PAI-2 or PAI-3, angiostatin or endostatin, interferon-alpha, -beta or -gamma, interleukin 12, platelet factor 4, thrombospondin-1 or -2, TGF-beta, TNF-alpha, vascular endothelial cell growth inhibitor (VEGI). In the meaning of the invention, the component I) may represent one or more nucleotide sequences that encode one or more identical or different, directly or indirectly, proliferatively or cytotoxically active proteins. Preferred are combinations of proteins, which have an additive or synergistic effect. Additive or synergistic effects can for instance be expected for the following combinations of differently active proteins: cytotoxic proteins and proapoptotic proteins, enzymes and cytotoxic and/or proapoptotic proteins, antiangiogenetic proteins and cytotoxic and/or proapoptotic proteins, inductors of inflammations and enzymes or cytotoxic, proapoptotic and/or antiangiogenetic proteins.

Component II).

Component II) is a nucleotide sequence that encodes at least one blood plasma protein under the control of an activation sequence that can be activated in the microorganism. Preferred are human blood plasma proteins, namely those, which have an average dwell time in the blood of more than 24 hours. To these belong in particular for instance albumin (nucleotide 1-2258; Hinchliffe et al., EP 0248637-A, Sep. 12, 1987), transferrin (nucleotide 1-2346; Uzan et al., Biochem. Biophys. Res. Commun. 119:273-281, 1984; Yang et al., PNAS-USA 81:2752-2756, 1984), ceruloplasmin (Baranov et al., Chromosoma 96:60-66, 1987), haptoglobin (nucleotide 1-1412; Raugei et al., Nucleic Acids Res. 11:5811-5819, 1983; Yang et al., PNAS-USA 80:5875-5879, 1983; Brune et al., Nucleic Acids Res. 12:4531-4538, 1984), hemoglobin alpha (nucleotide 1-576; Marotta et al., PNAS-USA 71:2300-2304, 1974; Chang et al., PNAS-USA 74:5145-5149, 1977), hemoglobin beta (nucleotide 1-626; Marotta et al., Prog. Nucleic Acid Res. Mol. Biol. 19:165-175, 1976; Marotta et al., J. Biol. Chem. 252:5019-5031, 1977), alpha2-macroglobulin (nucleotide 1-4599; WO 9103557 A, 21/3/1991). Thereto belong, however, other blood plasma proteins, too, such as alpha-1-lipoprotein, alpha-2-lipoprotein, beta-1-lipoprotein. The expression of at least one of these plasma proteins by the microorganism according to the invention has as a consequence that the microorganism is received after systemic administration—in particular after injection into the blood circulation system—to a lower degree by phagocytosing cells, thus can stay longer in the blood and can be enriched in the tumor vessel system or in the vessels of a chronic inflammation.

Component III).

Component III) is a nucleotide sequence that encodes a cell-specific ligand under the control of an activation sequence that can be activated in the microorganism. The specificity of this ligand depends on the kind of the proliferative disease, for which the microorganism is used, and on the cells or the tissue, with which component I) is to be brought into contact in the microorganism, in order to achieve the therapeutic effectivity. For instance, in tumor diseases, ligands with specificity for tumor cells are used, i.e. for tumor-associated or tumor-specific antigens or tumor endothelium cells or for tissue cells, from which the respective tumor originates, for instance for cells of the thyroid gland, the prostate, the ovary, the mammary, the kidney, the tunica mucosa gastris, the nevi, the cervix, the vesica urinaria; for chronic inflammations, cellular autoimmune diseases and rejections of transplanted organs, ligands either with specificity for macrophages, dendritic cells, T lymphocytes or for activated endothelium cells. Such ligands are for instance specific antibodies or antigen-binding fragments of these antibodies, growth factors, interleukins, cytokines or cell adhesion molecules selectively binding to tumor cells, to leukemia cells, to tumor endothelium cells, to tissue cells, to macrophages, dendritic cells, T lymphocytes or to activated endothelium cells.

Component IV).

Component IV) is a nucleotide sequence that encodes a transport system, which permits the expression of the expression products of components I), II) and/or III) on the outer surface of the microorganism. The respective component can as an option either be secreted or expressed on the membrane of the microorganism, i.e. membrane-bound. Components II) and III) are preferably expressed membrane-bound. Such transport systems are for instance the hemolysin transport signal of E. coli (nucleotide sequence containing hlyA, hlyB and hlyD under the control of the hly-specific promoter, Gentschev et al., Gene 179:133-140, 1996). The following transport signals can be used: for the secretion, the C-terminal hlyA transport signal, in presence of hlyB and hlyD proteins; for the membrane-bound expression, the C-terminal hlyA transport signal, in presence of the hlyB protein; the hemolysin transport signal of E. coli (nucleotide sequences containing hlyA, hlyB and hlyD under the control of a not hly-specific bacterial promoter); the transport signal for the S-layer protein (Rsa A) of Caulobacter crescentus; for the secretion and for the membrane-bound expression, the C-terminal RsaA transport signal (Umelo-Njaka et al., Vaccine 19:1406-1415, 2001); the transport signal for the TolC protein of Escherichia coli (the TolC protein was described by Koronakis et al., Nature 405:914-919, 2000) and by Gentschev et al., Trends in Microbiology 10:39-45, 2002)); for the membrane-bound expression, the N-terminal transport signal.

Component V).

Component V) is a nucleotide sequence that encodes at least one lytic for a protein, which is expressed in the cytosol of a mammalian cell and lyses the microorganism for the release of the plasmids in the cytosol of the host cell. Such lytic proteins (endolysins) are for instance Listeria-specific lysis proteins, such as PLY551 (Loessner et al., Mol. Microbiol. 16:1231-41, 1995), the Listeria-specific holin under the control of a listerial promoter. A preferred embodiment of this invention is the combination of different components V), for instance the combination of a lysis protein with a holin.

Component VI).

Component VI) represents an arbitrary activator sequence, which controls the expression of component I). For the expression of component I) on the outer surface of the microorganism, component VI) is one of activations sequences that can be activated in the bacterium and that is known to the man skilled in the art. Such activation sequences are for instance constitutively active promoter regions, such as the promoter region with ribosomal binding site (RBS) of the beta-lactamase gene of E. coli or of the tetA gene (Busby and Ebright, Cell 79:743-746, 1994), promoters that can be induced, preferably promoters that become active after reception in the cell. To the latter belongs the actA promoter of S. monocytogenes (Dietrich et al., Nat. Biotechnol. 16:181-185, 1998) or the pagc promoter of L. monocytogenes (Bumann, Infect. Immun. 69:7493-7500, 2001). Preferred are activator sequences, which, after release of the plasmids of the bacterial carrier in the cytosol of the target cell, are activated in this cell. For instance, the CMV enhancer, the CMV promoter, the SV40 promoter or any other promoter or enhancer sequence known to the man skilled in the art can be used. Preferred are further cell-specific or function-specific activator sequences. The selection of the cell-specific or function-specific activator sequence depends on the cell or the tissue, wherein the bacterial carrier or the plasmids released from the bacterial carrier are to express component I). Such activator sequences are for instance tumor cell-associated activator sequences (thereto belong activator sequences of the genes for midkine, GRP, TCF-4, MUC-1, TERT, MYC-MAX, surfactant protein, alpha-fetoprotein, CEA, tyrosinase, fibrillary acidic protein, EGR-1, GFAP, E2F1, basic myelin, alpha-lactalbumin, osteocalcin, thyroglobulin and PSA (McCormick, Nature Reviews Cancer 1:130-141, 2001), endothelium cell-specific activator sequences of the genes for proteins, which are preferably expressed by endothelium cells (Sedlacek, Critical Reviews in Oncology/Hematology 37:169-215, 2001), such as VEGF, von Willebrand factor, brain-specific endothelial glucose-1 transporter, endoglin, VEGF receptors, in particular VEGF-R1, VEGF-R2, and VEGF-R3, TIE-2, PDECGF receptors, B61, endothelin-1, endothelin-B, mannose 6-phosphate receptors, VCAM-1 and PE-CAM-1, activator sequences of the genes for proteins, which are preferably expressed in such tissue cells from which the tumor cells of a patient originate (thereto belong proteins expressed in cells of the breast tissue (for instance MUC-1, alpha-lactalbumin), the thyroid gland (for instance thyroglobulin), the prostate (for instance kallikrein-2, androgen receptors, PSA), the ovary, the nevi (for instance tyrosinase), and the kidney, activator sequences of the genes for proteins, which are expressed in macrophages, dendritic cells or lymphocytes, such as interleukins, cytokines, chemokines, adhesion molecules, interferons, receptors for interleukins, cytokines, chemokines, or interferons, activator sequences, which are activated by hypoxia, such as the activator sequence for VEGF or for erythropoietin.

The insertion of components I) to VI) into the microorganisms is made by molecular biological methods known to the man skilled in the art. For instance, for the use of bacteria as carriers, the man skilled in the art is familiar with how the components are inserted into suitable plasmids, and how these plasmids are introduced into the bacteria.

According to the present invention, these microorganisms are administered to a patient for the prophylaxis or therapy of a proliferative disease, such as a tumor, a leukemia, a chronic inflammation, an autoimmune disease or the rejection of an organ transplant. For treating such a disease, the microorganisms according to the invention are administered in a suitable preparation locally or systemically, for instance into the blood circulation, into a body cavity, into an organ, into a joint or into the connective tissue. In order, with systemic administration, in particular with administration into the blood circulation, to reduce the undesired reception of the microorganisms by the so-called reticuloendothelial system beyond the effect of component II) and to extend the blood dwell time of the microorganisms, the microorganisms can be suspended in a solution of substances, which have a long blood dwell time. To the suspension follows an incubation. The suspension and incubation of the microorganisms can for instance take place in blood plasma or blood serum. The suspension and incubation is however preferably performed in solutions of substances or solutions of mixtures of substances, which have a long blood dwell time. To these substances belong for instance albumin, transferrin, prealbumin, hemoglobin, haptoglobin, alpha-1-lipoprotein, alpha-2-lipoprotein, beta-1-lipoprotein, alpha-2-macroglobulin, polyethylene glycol (PEG), PEG conjugates with natural or synthetic polymers, such as polyethylene imine, dextran, polygeline, hydroxyethyl starch.

By the suspension and incubation in such a solution, an adsorption of the substances to the surface of the microorganisms according to the invention takes place. A coating of the microorganisms with these substances can however also be achieved by conjugation. The methods of the conjugation are summarized in Sedlacek et al., Contributions to Oncology 32:1-132, 1988.

The coating by adsorption takes place for instance by suspension of the microorganisms in a solution preferably containing 0.1 to 50% of the coating substances over a period of time of preferably 10 minutes to 24 hours and a temperature of preferably 4 degrees Celsius.

According to the invention, as microorganisms, preferably bacteria are used, the virulence of which has been reduced. Further preferred are bacteria selected from a group containing Escherichia coli, Salmonella enterica, Yersinia enterocolitica, Vibrio cholerae, Listeria monocytogenes, Shigella.

Microorganisms in conjunction with the invention are further membrane envelopes, so-called ghosts, of live or existing microorganisms. Such membrane envelopes are for instance produced according to EPA 0540525.

Subject matter of the invention are medicament preparations containing the microorganisms according to the invention and the use of this medicament for the prophylaxis and/or therapy of a proliferative disease. A proliferative disease in the meaning of the present invention is a disease with an escalating or uncontrolled cell proliferation, for instance a tumor disease such as a carcinoma or a sarcoma, a leukemia, a chronic inflammation, an autoimmune disease or the rejection of an organ transplant. For the prophylaxis or therapy of a disease, the microorganisms according to the invention are locally or systemically administered to a patient in the medicament preparation in a dose of preferably 100 germs to 100 million germs.

The term enveloped means that on the outside of the membrane of the microorganism, a multitude of identical or different molecules (expressed and/or selected according to one or more of features I) to III)), as described above, can be provided, the geometric coverage rate being between 0.001 and 1, in particular between 0.01 and 1, for instance between 0.1 and 1. The geometric coverage rate can be calculated from the ratio of the total area of all molecules, in a radial (related to a center of the microorganism) projection into the surface of the microorganism, divided by the surface area of the microorganism. Usually, as a simplification, a spherical surface of the microorganism is assumed, and the calculation is based on the volume of the microorganism. The feature “enveloped” is facultative, if applicable.

### EXAMPLES OF EXECUTION

### Example 1

### Construction of a Bacteria Strain for the Membrane-Bound Expression of Human Albumin and Beta-glucuronidase

In this example, the way to the bacteria strain St21-bglu is described. This attenuated Salmonella typhi Ty21a strain (carrier approved for human use) expresses by means of the hly secretion machinery of E. coli membrane-bound fusion proteins of human beta-glucuronidase and hlyA and human albumin and hlyA. The construction is based on the already published plasmids pMOhlyl (Gentschev et al., Behring Inst. Mitt. 57-66, 1994) and pGP704 (Miller and Mekalanos, J. Bacteriol. 170:2575-2583, 1988). The strain permits by passive targeting (Bermudes et al., Adv. Exp. Med. Biol. 465:57-63, 2000) an enrichment of beta-glucuronidase at the tumor and thus a fission restricted to the tumor tissue of prodrugs to be activated by beta-glucuronidase.

A membrane-bound expression can take place in salmonellae by fusion of the protein to the C-terminus of the hlyA secretion protein in presence of the hlyB protein, however in absence of a completely functional hlyD protein. However, the hlyD must not completely be missing, since otherwise there will not be generated a connection between the secretion machinery and the TolC protein of the outer membrane (Spreng et al., Mol. Microbiol. 31:1596-1598, 1999). In these examples one of the possible modifications of the hlyD protein for the membrane-bound expression is indicated. First the vector pMOhly DD is constructed, wherein no functional hlyD protein is produced. For this purpose, part of the hlyD gene is removed from the vector pMOhlyl by the endonucleases DraIII and ApaI. After the restriction digestion, the ends are digested by 3′-5′ exonuclease, and the 10,923 bps fragment is religated. Subsequently the beta-glucuronidase gene is cloned into this vector in-frame to the hlyA gene. For this purpose, the cDNA of bglu (GenBank Accession (Gb): M15182) from a cDNA bank was amplified with the following primers by polymerase chain reaction (PCR):

| | |
|---|---|
| bglu 5′: | ATGCATTGCAGGGCGGGATGCTGTACC |
| bglu 3′: | ATGCATAAGTAAACGGGCTGTTTTCCAAAC |

The regions being complementary to the cDNA of beta-glucuronidase are underlined, the information for the generated NsiI position is in italics (this kind of representation will also be used in the following; the oligonucleotide sequences are shown here, as in the following, as 5′-3′). The primers are selected such that the gene is amplified without the signal sequence. The product (1,899 bps) is subcloned with a suitable PCR cloning kit, and then the ≈1,890 bps fragment is extracted via NsiI digestion. Subsequently, the NsiI fragment is cloned into the NsiI-cut vector pMOhly DD. This results in the vector pMO DDbglu (FIG. 1). (When the NsiI fragment is cloned into the NsiI-cut vector pMOhlyl, the plasmid pMO bglu is obtained permitting a secernation of the fusion protein). In the second part the integration vector for the chromosomal integration of the albumin hlyA fusion is produced. In a first step, the vector pMOhly alb is produced. This vector being based on pMOhlyl carries a fusion of the albumin cDNA with the hlyA gene. For cloning, the cDNA of the albumin gene (Gb: A06977) from a commercially available cDNA bank is amplified by means of PCR and the following primers generating NsiI:

5′: ATGCATGGGTAACCTTTATTTCCCTTC
3′: ATGCATAGCCTAAGGCAGCTTGACTTG-

The 1,830 bps fragment is subcloned and then cut with NsiI. The 1,824 bps fragment is now ligated in NsiI-digested pMOhlyl. The completed plasmid pMOhly alb thus expresses hlyB, hlyD and a fusion protein from albumin and hlyA. For experiments regarding the dwell time, the NsiI fragment can alternatively also be inserted into the vector pMO DD, this vector has the name pMO DDalb. In the further course, a modification of the already described cloning strategy is used for the integration in the salmonella chromosome (Miller et Mekalanos, J. Bacteriol. 170:2575-2583, 1988). For this purpose, first the aroA gene of salmonella was cloned into the vector pUC18 (PCR with the following primers:

| | |
|---|---|
| primer 5′: | ATGGAATCCCTGACGTTACAACCC, |
| primer 3′: | GGCAGGCGTACTCATTCGCGC |

blunt cloning of the 1,281 bps fragment into the HincII interface of pUC18). Subsequently, a 341 bps fragment located in aroA was removed by HincII digestion and subsequent religation. This vector was called pUC18 aroA′. Then the alb-hlyA fusion gene was cloned together with the promoter sequence located on pMOhly into the vector pUC18aroA′. For this purpose, the vector pMOhly alb is digested with AacII and SwaI and then treated with a 3′-5′ exonuclease. The 3,506 bps blunt fragment is extracted and ligated in HincII-digested pUC18aroA′. This produces the vector pUCaro-alb. Now, the alb-hlyA fragment flanked by aroA is cloned with all the activator sequences from the vector pUCaro-alb into the vector pGP704. For this purpose, pUCaro-alb is digested with HindIII and then treated with 5′-3′ exonuclease (blunt). Subsequently, EcoRI digestion is performed, and the 4,497 fragment is ligated into the EcoRI/EcoRV (blunt) digested vector pGP704 (EcoRI/RV fragment: 6,387 bps). The integration vector pGParo-alb (FIG. 2) is obtained. The vector is transformed into the E. coli strain SM101pir. This strain permits the vector to replicate, since it forms the P protein necessary for replication. The vector is now transferred via conjugation into the acceptor strain Salmonella typhi Ty21a not permitting a replication of the vector. Therefore, by tetracycline selection, only those bacteria are selected that have integrated the vector chromosomally. The verification of the cytoplasmic albumin production takes place by Western blot analysis of the bacterium lysate. This strain St21-alb expresses the alb-hlyA fusion, but can neither secern nor express it on the membrane in this form. For this purpose, for the membrane-bound expression, in addition a plasmid with functional hlyB (as pMO DDbglu) or functional hlyB and hlyD (as pMO bglu) needs to be present.

In this example, the plasmid pMO DDbglu with the strain St21-alb is used. This results in the strain St21-alb pMO DDbglu expressing by means of the hly secretion system human albumin as well as human beta-glucuronidase on the membrane. This strain can then be used for the prodrug conversion in the meaning of the patent.

### Example 2

### Construction of a Bacteria Strain Enveloped with Albumin-hlyA Fusion for Supplying the Genetic Information of Human Beta-glucuronidase.

The bacteria strain described in this example is intended to supply by means of the passive targeting DNA that encodes human beta-glucuronidase for tumor cells, which are then to be expressed in the tumor cells. In order to obtain a strain being particularly easy to handle, in this example a slightly modified strain as in Example 1 is used for the membrane expression of albumin. The gene that encodes albumin-hlyA as well as the information for hlyB is to be chromosomally integrated. Thereby, this strain expresses constitutively membrane-bound albumin.

For this purpose, the vector pMOhly alb described above is digested by BsrBI and EcoRI and then treated with 5′-3′ exonuclease. This digestion produces a 5,815 bps fragment with blunt ends containing the complete prokaryontic activation sequence and the genes hlyC, alb-hlyA and hlyB, not however hlyD. This fragment can now bluntly be inserted into the HincII interface of the vector pUC18aroA∝0 described above. Thereby the vector pUCaro-alb-B is obtained. By an EcoRI-NruI digestion, the 6,548 bps fragment can again be inserted into the EcoRI-EcoRV-digested vector pGP704 (FIG. 3). The further procedure (replication and integration in S. typhi Ty21a) corresponds to the above strategy. The resulting strain St21-alb-B expresses constitutively membrane-bound albumin-hlyA fusion protein. If a vector that encodes hlyD is transfected, the albumin-hlyA fusion protein is secerned. The plasmid for supplying the DNA that encodes beta-glucuronidase is based on the commercially available vector pCMVbeta (Clontech). For the construction, first a fusion of the bglu gene with a secretion signal must be used. In this example, the signal peptide of the tPA precursor molecule is to be used. This signal peptide permits a particularly efficient production and secretion of fusion proteins. For cloning the fusion, in a first step the 5′ UTR of the tPA cDNA (Gb E02027) is amplified up to the end of the region that encodes the signal peptide with the following primers via PCR (amplification with blunt generating polymerase):

5′: GCGGCCGCAGGGAAGGAGCAAGCCGTGAATTT
3′: AGCTTAGATCTGGCTCCTCTTCTGAATC

The generated 166 bps fragment is ligated into the HindIII-digested, 51-3′ exonuclease-treated commercially available vector pcDNA3 (Invitrogen). The ligation is made in the forward orientation. Thereby, the region that encodes tPA signal sequence can completely be cut out via a NotI digestion from the generated plasmid pCDNAtp. This 237 bps fragment is now ligated with the 3,760 bps fragment of the vector pCMVbeta after NotI digestion (contains vector backbone). The generated plasmid pCMVtp (3,972 bps) can now be used for the expression of heterologous fusion proteins. For the generation of the plasmid pCMV bglu, a bps fragment of the bglu (Gb M15182) gene (without sequence for signal peptide) from a suitable cDNA bank is amplified with the following primers generating SpeI:

5′: ACTAGTCAGGGCGGGATGCTGTACCCCCAG
3′: ACTAGTCTTGCTCAAGTAAACGGGCTGTTTTC.

After SpeI-digestion, the 1,899 bps fragment is ligated into the SpeI-digested vector pCMVtp. The generated plasmid pCMVtp bglu encodes now an N-terminal fusion of the tPA signal peptide with the region of the mature protein of beta-glucuronidase. After determination of the correct position, the plasmid pCMVtp bglu (FIG. 4) is transformed into the strain St21-alb-B. This strain permits now a supply of the DNA to the tumor tissue by means of passive targeting, and the expression of the DNA by transfected tumor cells permits then a conversion of suitable prodrugs.

### Example 3

### Construction of a Strain Enveloped with Albumin-TolC Fusion with Membrane-Bound Expression of the Extra-Cellular Domain of fas and Supply of an Enzyme Converting Prodrug

The strain shown in this example unites the features shown in Example 2 with a specific targeting at (tumor) cells expressing fas ligand (fasL). It is possible, with this strain, to specifically attack fasL-expressing tumor cells, such as in certain breast tumors (Herrnring et al., Histochem. Cell. Biol. 113:189-194, 2000). fasL expression by tumor cells was postulated as a potential mechanism for immune escape, since these cells can eliminate actively attacking, fas-expressing lymphocytes (Muschen et al., J. Mol. Med. 78:312-325, 2000). With the strain shown here, these tumor cells being very problematic for a therapy can specifically be attacked and then eliminated by an apoptosis-independent mechanism. The carrier strain is based in this example on a fusion of albumin with the TolC protein of E. coli. Thereby, a membrane-bound expression of albumin is achieved. The membrane-bound expression of the extracellular domain of fas takes place via the plasmid pMOhlyDD, and for the supply the plasmid pCMV-bglu described above is used. The first step comprises the generation of the carrier strain expressing TolC albumin. First the gene for the fusion protein is generated, and then this gene is integrated, according to the above examples, via successive cloning in pUCaroA′ and pGP704 into the salmonella genome. The TolC gene for E. coli, including the natural promoter, is present in the plasmid pBRtolC. This was amplified by means of the following primers generating SalI from the vector pAX629 (contains tolC gene, region in the vector corresponds to Gb X54049 pos. 18-1914):

| | |
|---|---|
| 5′tol: | TAACGCCCTATGTCGACTAACGCCAACCTT, |
| 3′tol: | AGAGGATGTCGACTCGAAATTGAAGCGAGA. |

The 1,701 bps fragment was inversely ligated after fission with SalI into the SalI interface of the vector pBR322 (Gb J01749), thus the tet gene being interrupted. Due to the known crystal structure of TolC (Koronakis et al., Nature 405:914-919, 2000), the insertion of heterologous DNA into the singular KpnI interface in the tolC gene permits the expression of the encoded heterologous fusion protein in an extracellular loop on the outer membrane. For the expression of albumin, the albumin gene is amplified from the cDNA (Gb A06977) by means of the following primers generating KpnI:

5′: GGTACCCGAGATGCACACAAGAGTGAGG
3′: GGTACCTAAGCCTAAGGCAGCTTGACTTGC.

After KpnI digestion of the 1,770 bps fragment, the DNA can be inserted into the KpnI-cut vector pBRtolC. The reverse orientation (in frame to tolC) results then in the vector pBRtolC-alb. The gene for the tolC-albumin fusion is ligated now in reversed orientation via EcoRV and PshAI (fragment 3,970 bps) into the HincII interface of the vector pUCaroA′. The obtained vector pUCaro-alb-tol (7,596 bps) is now linearized with HindIII, treated with 5′-3′ exonuclease and then digested with EcoRI. The 4,961 bps fragment is then inserted into the EcoRI-EcoRV-digested vector pGP704 (FIG. 5). After conjugation (according to Example 1) the strain St21-tol-alb is obtained. Now the plasmid is used for the membrane-bound expression of a fas (CD95)-hlyA fusion protein by means of the hlyB component of the E. coli type I secretion machinery. For this purpose, first the section that encodes the extracellular region of the fas gene (Gb: M67454) is amplified with the following primers generating NsiI:

5′: ATGCATTATCGTCCAAAAGTGTTAATGC
3′: ATGCATTAGATCTGGATCCTTCCTCTTTGC.

The 477 bps fragment is digested with NsiI and inserted into the NsiI-digested vector pMOhly DD in frame to the hlyA gene. The obtained vector pMO DD-fas (FIG. 6) thus produces after transformation into a salmonella strain a membrane-bound fas fragment, which with suitable folding can bind to fasL-expressing cells. Thus, these salmonellae can be enriched at fasL-expressing cells, such as tumor cells.

For killing the fasL tumor cells, now the plasmid pCMV bglu (Example 2) is also transfected into the salmonellae. Thereby, as in the above example, after expression of the beta-glucuronidase by tumor cells, a prodrug-drug-mediating tumor therapy is possible. The better effectiveness of this example compared to the previous example depends in a decisive way on the correct folding of the extracellular domain of fas. In lieu of fas, fasL-specific fab fragments of monoclonal antibodies (which can correctly be folded in bacteria) can be used in the same approach as described here. This example shows that by means of this technique, the construction of strains with nearly any cell specificity is possible via the use of suitable specific fab fragments.

### LEGEND OF THE FIGURES

FIG. 1: vector pMO Dbglu

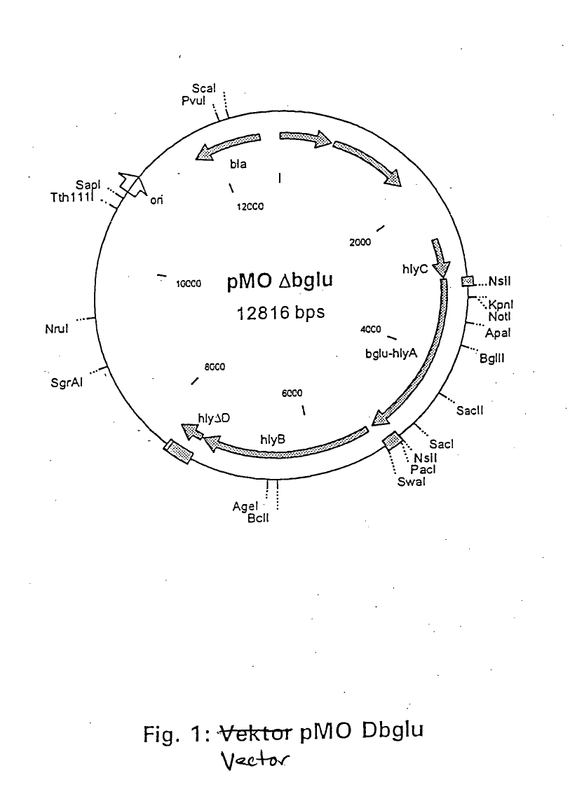

FIG. 2: vector pGParoalb

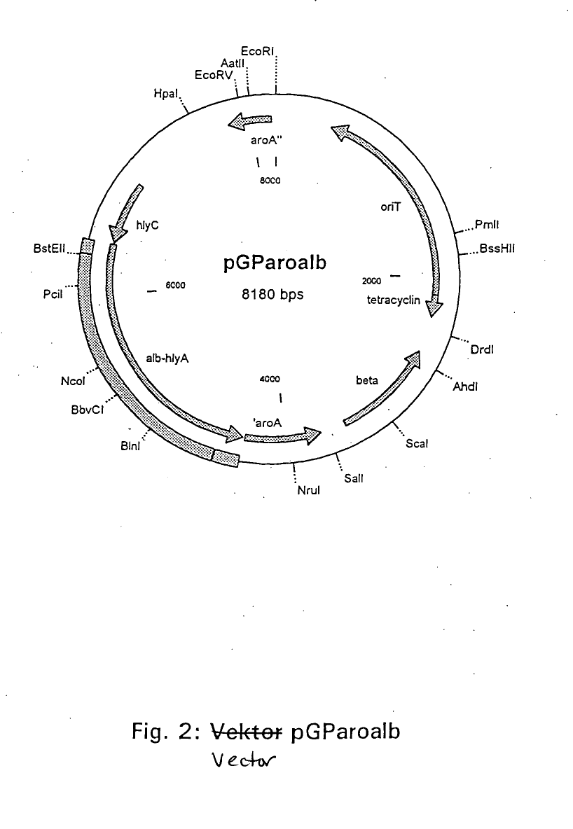

FIG. 3: pGParo-alb-B

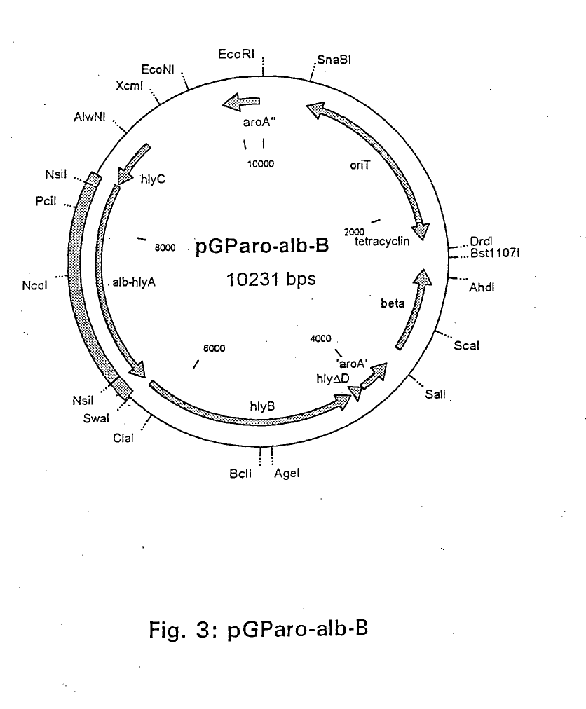

FIG. 4: pCMVtp bglu

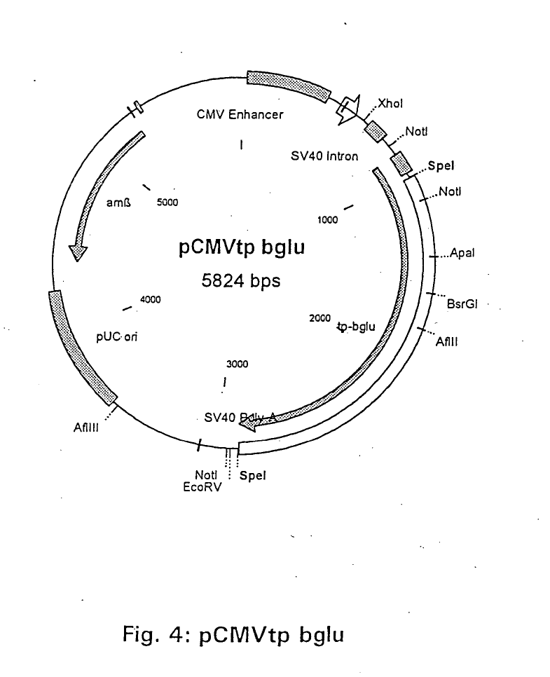

FIG. 5: pGParo-alb-tol

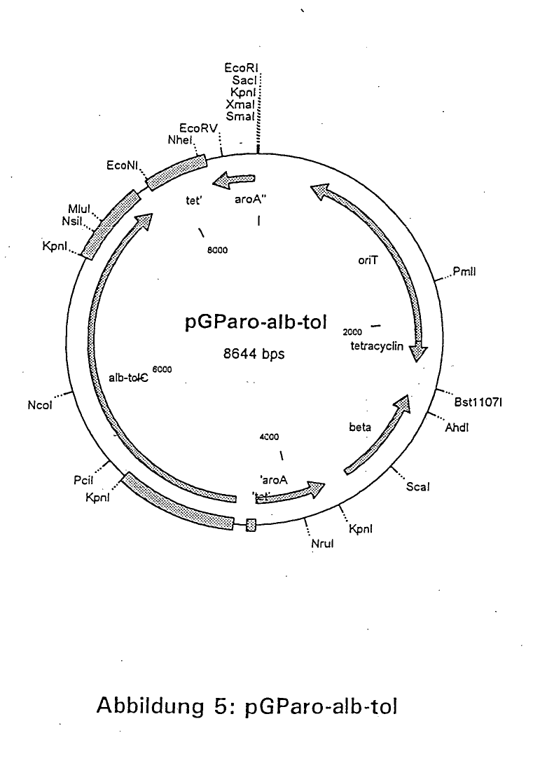

FIG. 6: pMO DD-fas

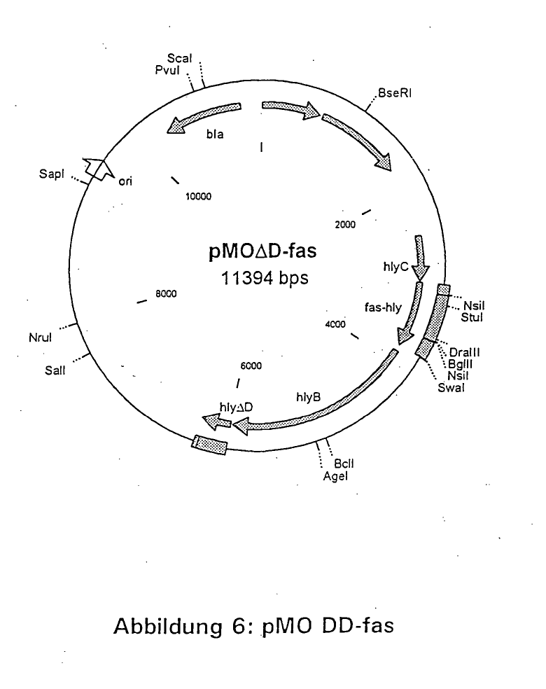

1. A microorganism in whose genome the following components are inserted and can be expressed:
a) a nucleotide sequence that encodes a direct or indirect antiproliferative or cytotoxically active expression product or a plurality of said expression products,
b) a nucleotide sequence that encodes for a blood plasma protein under the control of an activation sequence that can be activated in the microorganism, or that is constitutively active,
c) a nucleotide sequence that encodes for a cell-specific ligand under the control of an activation sequence that can be activated in the microorganism, or is constitutively active,
d) a nucleotide sequence for a transport system that induces expression of the expression products of components a) and b) and optionally c) on the outer surface of the microorganism or that induces secretion of the expression products of component a) and expression of compenent b) and optionally c) and that is preferably constitutively active,
e) a nucleotide sequence for a protein used for lysis of the microorganism in the cytosol of mammalian cells and for the intracellular release of plasmids with at least one or more components a) and f) contained in the lysed microorganism, and
f) an activation sequence that can be activated in the microorganism, or that is tissue cell-specific, tumor cell-specific, function-specific or not cell-specific, for expressing component a),
wherein any of components a) to f) is present either once or several times, and are either identical or different.

2. The microorganism according to claim 1, wherein the microorganism is a virus, a bacterium or a monocellular parasite.

3. The microorganism according to claim 1 or 2, wherein the virulence of the microorganism is reduced.

4. The microorganism according to claim 1, wherein the microorganism is a gram-positive or gram-negative bacterium.

5. The microorganism according to claim 1, selected among a group consisting of Escherichia coli, Salmonella, Yersinia enterocolitica, Vibrio cholerae, Listeria monocytogenes, and Shigella.

6. The microorganism according to claim 1, wherein the microorganism is the envelope of a bacterium.

7. The microorganism according to claim 1, wherein component a) encodes at least one protein selected from the group consisting of interferons; interleukins; proapoptotic proteins; antibodies and antibody fragments, which act inhibitingly on or cytotoxically for an immune cell, a tumor cell or for cells of the tissue, from which the tumor originates; antiproliferatively active proteins; cytotoxic proteins; inductors of an inflammation, in particular interleukins, cytokines or chemokines; viral, bacterial enzymes or enzymes that originate from a yeast, a mollusk, a mammal or man for the activation or fission of an inactive pre-stage of a cytostatic substance into the cytostatic substance; fusion products from a cell-specific ligand and an enzyme; and inhibitors of the angiogenesis.

8. The microorganism according to claim 1, wherein component b) encodes at least one blood plasma protein selected from a group consisting of albumin, transferrin, haptoglobin, hemoglobin, alpha-1-lipoprotein, alpha-2-lipoprotein, beta-1-lipoprotein and alpha-2-macroglobulin.

9. The microorganism according to claim 1, wherein component c) encodes at least one ligand specific for a target organism selected from a group consisting of tumor cells; tumor endothelium cells; tissue cells, from which originates a tumor; activated endothelium cells; macrophages; dendritic cells; and lymphocytes.

10. The microorganism according to claim 1, wherein component c) encodes at least one ligand specific for a tissue cell type of tissues selected from a group consisting of thyroid gland, mammary, salivary gland, lymph gland, mammary, tunica mucosa gastris, kidney, ovary, prostate, cervix, vesica urinaria, and nevus.

11. The microorganism according to claim 1, wherein component d) encodes the hemolysin transport signal of Escherichia coli, the S-layer (Rsa A) protein of Caulobacter crescendus, or the ToiC protein of Escherichia coli.

12. The microorganism according to claim 1 wherein component e) encodes a lytic protein of gram-positive bacteria, lytic proteins of Listeria monocytogenes, PLY551 of Listeria monocytogenes or holin of Listeria monocytogenes.

13. The microorganism according to claim 1, wherein at least one substance is bound to the microorganism which has a long blood dwell time and which is selected among the group consisting of albumin, transferrin, prealbumin, hemoglobin, haptoglobin, alpha-1-lipoprotein, alpha-2-lipoprotein, beta-1-lipoprotein, alpha-2-macroglobulin, polyethylene glycol (PEG), PEG conjugates with natural or synthetic polymers, such as polyethylene imine, dextran, polygeline, hydroxyethyl starch and mixtures of these substances, wherein the binding of the substance or substances takes place by physisorption, chemisorption or covalently.

14. A plasmid or expression vector comprising components a) b) d) and f) and one or more of components c) and e).

15. A method for the production of an organism according to claim 1, wherein a plasmid or expression vector comprising components a) b) d) and f) and one or more of components c) and e, is produced, and a microorganism is transformed with this plasmid.

16. A pharmaceutical composition comprising a microorganism according to claim 1.

17. A pharmaceutical composition for the prophylaxis and/or therapy of a disease, which is caused by an uncontrolled cell division comprising a tumor disease comprising a prostate carcinoma, an ovary carcinoma, a mamma carcinoma, a stomach carcinoma, a kidney tumor, a thyroid gland tumor, a melanoma, a cervix tumor, a bladder tumor, a salivary gland tumor or a lymph gland tumor, a leukemia, an inflammation, an organ rejection, or an autoimmune disease, wherein the composition comprises the microorganism according to claim 1.

18. The composition of claim 17 wherein the composition is used for removal of a tumor as well as of healthy tissue, from which originates the tumor.

19. A method for the production of a pharmaceutical composition according to claim 16, wherein an enveloped microorganism according to claim 1 is prepared in a physiologically effective dose with one or more physiologically tolerated carrier substances for oral, intramuscular, intraveneous or intraperitoneal administration.
